(19) United States
(12) Patent Application Publication
(10) Pub. No.: US 2010/0273984 A1
(43) Pub. Date: Oct. 28, 2010

(54) METHOD FOR ENRICHING PHOSPHOPEPTIDES

(75) Inventors: Jan Petzel, Solingen (DE); Kerstin Steinert, Langenfeld (DE); Udo Roth, Bonn (DE)

Correspondence Address: RANKIN, HILL & CLARK LLP, 23755 LORAIN ROAD, SUITE 200, NORTH OLMSTED, OH, 44070, US

(73) Assignee: QIAGEN GMBH, Hilden (DE)

(21) Appl. No.: 12/747,127

(22) PCT Filed: Dec. 29, 2008
(86) PCT No.: PCT/EP08/11129

§ 371 Date: Jun. 9, 2010

(30) Foreign Application Priority Data
Dec. 28, 2007 (DE) 10 2007 063 356.6

(51) Int. Cl. C07K 1/04; B32B 9/04

(52) U.S. Cl. 530/345; 428/704

(57) ABSTRACT

The invention relates to a method for enriching phosphopeptides. Said method is characterized in that a carrier is used which carries phosphate groups and/or phosphonate groups on the surface thereof. The phosphate groups and/or phosphonate groups are functionalized with zirconium ions and are bonded to the carrier by means of linker structures which have at least one alkyl chain containing at least 5 C atoms. Also disclosed are corresponding carriers and suitable kits for enriching phosphopeptides.

This invention relates to a method for enriching/isolating phosphorylated peptides and proteins (summarized hereinafter: phosphopeptides) from complex sample mixtures using specifically functionalized carrier materials.

The entirety of all proteins in a creature, a tissue, a cell, or a cell compartment under strictly defined conditions and at a particular time point is usually termed the proteome. The proteome is in an equilibrium between continuous new synthesis of proteins and simultaneous degradation of proteins which are no longer required. Hence, the proteome is, in contrast to the relatively static genome, subjected to continuous changes in its composition. These changes are controlled via complex regulatory processes.

The complexity of the cellular proteome increases exponentially once posttranslational modifications of proteins are included. The dynamic posttranslational modification of proteins is often decisive for the formation and regulation of protein structure and function. Currently, hundreds of different posttranslational modifications of proteins are known, of which phosphorylation represents by far the most prominent. Enzymatically catalyzed phosphorylation and dephosphorylation is an important regulatory element for the living cell. Organisms make use of reversible protein phosphorylation to control fundamental cellular processes such as signal transduction, the cell cycle, metabolism and also programmed cell death and gene expression. The transient and reversible phosphorylation of particular amino acids in proteins involved in these processes serves to stringently control activity, stability, localization, or interactions. A comprehensive analysis of phosphopeptides and the determination of phosphorylation sites is thus a prerequisite for an understanding of complex biological systems and, often, of causes of diseases as well.

Owing to the low amounts, the analysis and identification of phosphopeptides and the identification of phosphorylation sites must, as a rule, be effected by sensitive, mass spectrometric methods. These methods require typically the enzymatic cleavage of the phosphoprotein or phosphopeptide to be analyzed into fragments, mostly into tryptic peptides (obtainable by cleavage with trypsin). Phosphorylated amino acids occur, however, only in such peptides which contain the recognition sequences for the enzymes involved in the phosphorylation. However, as a rule, the proteins involved in regulatory processes are represented in the cell only at a relatively low abundance and hence are difficult to analyze, since peptides below a particular relative abundance in the peptide mixture can no longer be confidently detected. Moreover, the transient phosphorylation of proteins is rarely stoichiometric, so the phosphorylated form, as a rule, is present together with the unphosphorylated form. Accordingly, even when analyzing a phosphoprotein purified to homogeneity, phosphopeptides are present in admixture with unphosphorylated peptides of the same protein, which makes analysis difficult.

In order to identify phosphorylation sites in a protein, mass spectrometric methods are generally employed. After digestion of a protein/peptide or a protein/peptide mixture, the peptides thus recovered are identified by means of mass spectrometry. Since the phosphorylated peptides, however, tend to ionize not as well as unphosphorylated peptides, phosphopeptides are, as a rule, underrepresented in complex mixtures or even completely suppressed. Furthermore, stoichiometric effects make analysis difficult (see above).

Therefore, weak, small signals can disappear in the background noise, so low-abundance peptides—which, however, are often precisely of central significance—cannot possibly be detected without prior enrichment. Therefore, the phosphopeptides to be studied are, as a rule, initially enriched in order to prepare them for mass spectrometric analysis and in order to substantially avoid suppression effects. It is estimated that, in the human proteome, about 100 000 potential phosphorylation sites are encoded in the primary sequence of corresponding proteins; of these sites, however, only about 2000 could be identified so far.

Strategies for selectively and efficiently enriching phosphorylated peptides from proteolytic extracts of phosphorylated proteins with a high yield are thus an important component of a comprehensive analysis of the phosphoproteome. Various methods have been developed for this purpose, including the use of titanium oxide, IMAC methods, and phosphoramidite-based methods. With each of these methods, however, only particular subpopulations of phosphopeptides can be enriched, while others are not enriched (see, for example, Reproducible isolation of distinct, overlapping segments of the phosphoproteome; Bodenmiller et al., Nature Methods 4 (3), 2007, 231-237). For example, IMAC surfaces prefer multiphosphorylated peptides, whereas TiO₂ preferentially enriches monophosphorylated peptides.

Furthermore, in methods known in the prior art, nonspecific interactions also take place; for example, Fe-IMAC also binds acidic peptides, which is disadvantageous for the specificity of enrichment.

Recently, the employment of ZrO₂ as an alternative to TiO₂ has become known. However, the binding of phosphopeptides to ZrO₂, in comparison with TiO₂, has proved to be less specific, which is why the addition of additives is recommended. Also, elution often causes problems.

Zhou et al. (Zirconium phosphonate-modified porous silicon for highly specific capture of phosphopeptides and MALDI-TOF MS analysis, J. Prot. Res., 2006, 5, 2431-2437) disclose the employment of porous silicate surfaces modified with zirconium phosphonate, wherein the phosphonate group is directly coupled to the porous silicon. Zhou et al. were able to show, with this carrier-linker structure, a relatively specific enrichment of phosphopeptides compared with conventional Fe-IMAC methods. In comparison with Fe-IMAC methods, an improved specificity with comparable selectivity was achieved. The selectivity, however, could not be improved, i.e., only some of the phosphopeptides present in the sample were detected. It is, however, desirable to analyze the largest possible number of different phosphopeptides in a complex mixture in order that, in particular, even the proteins in low amounts may be captured as completely as possible.

Owing to the distinct disadvantages of the individual methods, a combination of different methods is therefore generally recommended in order to be able to analyze a broad spectrum of phosphopeptides.

A reproducible enrichment method for phosphorylated peptides for the analysis of the phosphoproteome should, because of the often low abundance of phosphoproteins and because of the substoichiometric occurrence of phosphorylation, deliver a highly quantitative yield of the corresponding phosphopeptide so that low-abundance phosphopeptides can also be detected and are thus available for analysis. At the same time, the enrichment method should deliver quantitative purity in order to allow, despite the abovementioned stoichiometric effects, direct analysis of the phosphopeptides in the sample.

It is an object of the present invention to provide a method for enriching/isolating phosphopeptides from a sample that ensures enrichment of the broadest possible spectrum of phosphopeptides.

This object is achieved in the present invention by a method for enriching phosphopeptides, characterized in that enrichment makes use of a carrier which carries on its surface phosphate and/or phosphonate groups which are functionalizable with zirconium ions, wherein the phosphate and/or phosphonate groups are bound to the carrier via linker structures and the linker structures have at least one alkyl chain which has at least 5 carbon atoms. The zirconium ions become immobilized upon contact with the phosphate or phosphonate groups, providing a carrier surface to which phosphopeptides (i.e., phosphopeptides and phosphoproteins; the term phosphopeptides does not imply any size limitation) can become specifically bound and hence enriched.

The employment of carrier materials functionalized with zirconium ions (Zr⁴⁺) and phosphonate groups for enriching phosphopeptides was already known in the prior art. As explained above, with conventional methods a broad spectrum of phosphopeptides could, however, often not be enriched and hence analyzed, i.e., some of the phosphorylated peptides were not detected. In contrast to the prior art, long, flexible linker structures are employed in the method according to the invention in order to bind phosphate or phosphonate groups functionalizable with zirconium ions to the carrier. For this purpose, the linker structures have at least one alkyl chain which has at least 5, preferably ≧7, more particularly ≧10 carbon atoms. In an assembly (for example, as SAM), these long linker structures can, however, also form highly ordered structures which are rigid to crystalline. The alkyl chains can, however, also feature one or more groups and, for example, be interrupted by them, for example, polymer groups, sulfide groups, or disulfide groups. Appropriate groups can also be attached to the alkyl chain. The alkyl chain can therefore also have further groups, more particularly such groups as increase the flexibility of the phosphate and/or phosphonate groups. According to the invention, not only alkanethiols, but also, for example, dialkyl disulfides, dialkyl sulfides, and ethylene glycol alkanethiol derivatives can therefore be employed as linker structure. Examples are presented in detail below in connection with the coating method and are also valid in general in connection with the carrier according to the invention.

By employing the linker structures according to the invention, a flexible but ordered functionalized carrier surface is provided. Such a surface has proven to be advantageous, enriching the broadest possible spectrum of phosphopeptides and hence bringing them to analysis. The experimental data show that, with the method according to the invention or the carrier according to the invention, more different phosphopeptides can be detected than with methods known in the prior art. Therefore, with the method according to the invention, complex samples containing many different phosphopeptides (i.e., phosphopeptides and phosphoproteins) can also be analyzed; such samples would normally be unanalyzable or partially analyzable, since phosphopeptides of low abundance would go missing. With the method according to the invention, samples which, if need be, are even unpurified can be employed. Altogether, the method according to the invention allows, therefore, a good coverage of the proteome and is a valuable contribution to the art.

With the method according to the invention, a broad spectrum of phosphopeptides can therefore be enriched, with the phosphate/phosphonate-Zr⁴⁺ technology according to the invention enabling a high specificity and binding strength, which is in turn advantageous for the quality of phosphopeptide enrichment. With the method according to the invention, a highly specific and efficient enrichment of phosphopeptides is therefore possible. The mass spectrometric study shows little, if any, nonspecific binding, so most of the peaks identified in a mass spectroscopic study originated from peptides which are phosphorylated and hence of interest.

This difference, which is apparently due to the linker structures to be employed according to the invention, which are long and therefore flexible to a certain extent, is surprising because it was initially assumed that it would be essentially the metal compounds employed (here: zirconium ions) which would be significant for the binding properties of the carrier. With the present invention, it was shown, however, that the environment, more particularly the connection of functional phosphate/phosphonate-zirconium ion groups to the carrier, is also of decisive significance for the efficiency of the enrichment method. In order to achieve the greatest possible flexibility for the linker structure, the alkyl chain of the linker structure has preferably ≧10 or even ≧13 carbon atoms. The length and the resulting flexibility of the linker structure is believed to facilitate better interaction of the functional groups with the different phosphopeptides, causing more different phosphopeptides to bind. As explained, alkylthiols in assembly (for example, as SAM) can have in turn a strongly ordered and therefore rigid structure. If desired, the flexibility can be increased here by attaching, for example, polymer groups, such as PEG groups, to the alkyl chain in order to enable a flexible interaction of the phosphate or phosphonate groups functionalized with zirconium ions with the phosphopeptides to be enriched.

The present invention hence makes available a valuable instrument for enriching and analyzing phosphopeptides, which eases analysis of the proteome and complements the methods already known for such analysis.

In order to increase the flexibility of the linker structure further, it has, as explained, proven advantageous to integrate at least one inert polymer group in the linker structure, for example, in the alkyl chain, and/or to attach at least one inert polymer group to the alkyl chain. An example of such an inert polymer group is polyethylene glycol (PEG). Such a PEG group (EO4) was employed, for example, in the linker structure HS C11 EO4 CH₂CH₂—PO₃H₂, which is highly suitable for the purposes of the invention. This carries phosphonate groups.

A multiplicity of carriers can be employed with the method according to the invention, for example, plates, filters, small columns, polymer particles, magnetic particles, metal particles, silica particles, silica carriers, glass substrates, and coated substrates, such as MALDI carriers for example. As explained above, the phosphoproteome is preferably studied by means of MALDI.

Metals or metal surfaces can also be advantageously employed as carriers, examples being silver, copper, platinum, mercury, palladium, iron, and also iron oxides (γ-Fe₂O₃), and more particularly gold or gold surfaces. These are preferred when employing a MALDI carrier.

Accordingly, a MALDI carrier is preferably employed. MALDI substrates can be easily functionalized with the linker structures to be employed according to the invention. As a result, there is provided a tool for enriching phosphopeptides which enables, after immobilization of zirconium ions on phosphate or phosphonate groups, direct analysis of bound samples by means of MALDI. This facilitates use, since the user can apply the sample, which can even be unpurified if need be, on the MALDI chip functionalized with the linker groups according to the invention and the —PO₃Zr⁴⁺ or —PO₄Zr⁴⁺ group and begin directly with analysis. This is a huge advantage compared with the prior art, in which cleanup steps often had to be carried out before actual MALDI analysis, which, however, risked losing particular phosphopeptides present in small amounts during the cleanup steps upstream of the analysis.

The linker structure can be joined to the carrier by either convalent or noncovalent bonding. Examples of highly suitable linker structures, owing to their flexibility and yet ordered structure, are, for example, silanizations, SAMs, or Langmuir-Blodgett films which are provided with the phosphate or phosphonate groups according to the invention. Appropriate linker structures provide a surface structure which is flexible to a certain extent and simultaneously highly ordered.

As explained, both phosphate and phosphonate groups can be employed in order to immobilize zirconium ions at the carrier surface. Usually, the carrier endowed with phosphate and/or phosphonate groups is produced initially. Functionalization with zirconium ions is effected preferably just before the actual enrichment and hence just before the application of the sample. However, functionalization can also be effected in advance.

Methods for applying silane groups, SAMs, or Langmuir-Blodgett films are well known in the prior art. Langmuir-Blodgett linker structures are preferably bound via ionic or electrostatic bonds to the actual carrier, preferably a MALDI substrate. SAM linker structures can be bound, for example, via SH groups or disulfide groups to the carrier. Silane linker structures are, as a rule, bound by covalent bonds to the carrier. These are preferably Si—O or Si—C bonds.

Especially preferred is an embodiment in which the carrier employed is a gold-coated MALDI carrier which carries an SAM layer which is functionalized with PO₃Zr⁴⁺ or PO₄Zr⁴⁺ groups. As explained, the carrier is preferably provided initially only with the linker structure and the phosphate or phosphonate group. This carrier is then functionalized with Zr⁴⁺ ions before the actual analysis, making the carrier ready for enrichment. As explained, functionalization with zirconium ions is preferably effected just before the application of the actual sample. However, also conceivable are versions in which the carrier is loaded in advance with zirconium ions. Suitable carriers are, for example, made of stainless steel, silicon, or glass; these can also be coated with semiprecious metals, for example copper, and/or precious metals.

The enrichment/purification of phosphopeptides following functionalization of the carrier with zirconium ions is effected according to conventional methods known in the prior art, wherein customary wash and binding buffers can be employed. The wash and binding buffers usually employed work in a pH range of <3 in order to suppress nonspecific binding of acidic unphosphorylated peptides. ACN (acetonitrile) is often employed in the wash buffer in order to avoid possible hydrophobic interactions between hydrophobic peptides and the linkers.

Preferably, a MALDI carrier is employed which has a gold coating, wherein the gold layer is functionalized with the following SAMs: HS C11 EO4 CH₂CH₂—PO₃H₂ or HS—C₁₁—(OCH₂CH₂)₄—PO(OH)₂.

As can be recognized, a PEG group (EO4) is integrated in the alkyl chain or attached to the alkyl chain. The thiol group used to connect the linker structure to the carrier is followed by a chain of 11 carbon atoms, a PEG group (EO4), a further C2 grouping, and then the phosphonate group to which zirconium ions can be bound.

The present invention further provides a carrier for enriching phosphopeptides, characterized in that it carries on its surface phosphate and/or phosphonate groups which are functionalizable with zirconium ions, wherein the phosphate and/or phosphonate groups are bound via linker structures to the carrier and the linker structures have at least one alkyl chain which has at least 5, preferably at least 9, preferably at least 10 carbon atoms. As explained, the alkyl chain can also have other groups within the alkyl chain and therefore be “interrupted”, or alternatively, appropriate groups, such as polymer groups, more particularly PEG groups, can be attached to the alkyl chain (see above). According to the invention, not only alkanethiols, but also, for example, dialkyl disulfides, dialkyl sulfides, and ethylene glycol alkanethiol derivatives can therefore be employed. Examples are presented in detail below in connection with the coating method and also apply generally in connection with the carrier according to the invention.

The synthesis of such compounds, which have not only an alkyl chain but also other groups, preferably proceeds from an alkyl chain (from this, an SAM layer can be formed) and then attaches to it the PEG groups (or other groups if desired). As a result, an additional flexibilization of the phosphate and/or phosphonate group can be achieved.

As discussed at length above, such a carrier with linker structures which are long and therefore flexible to a certain extent is especially suitable for purifying a very wide spectrum of phosphopeptides, provided it is functionalized with zirconium ions. Advantageous improvements and refinements of such a carrier have already been described in detail above. We refer to our observations above.

In one embodiment, the carrier comprises bound phosphopeptides. Such a carrier arises, for example, as soon as the carrier according to the invention is employed for purifying and enriching phosphopeptides.

The present invention further provides a method for producing a carrier according to the invention for enriching phosphopeptides, characterized in that a carrier which has on its surface phosphate and/or phosphonate groups which are bound to the carrier via linker structures which have at least one alkyl chain having at least 5 carbon atoms is brought into contact with zirconium ions in order to generate on the carrier a phosphopeptide-binding, functional surface.

Suitable carrier materials have already been described above. In a preferred embodiment, magnetic particles are employed as carrier materials. By bringing the sample into contact with the magnetic carrier materials, which are modified with the linker structures according to the invention, the phosphopeptides bind specifically to the surface of the particles. The particles can then be easily separated from the remaining sample by applying an external magnetic field.

The magnetic particles can be, for example, polymers, for example polystyrene, or inorganic materials, for example silica, which are magnetizable by additives or coating with magnetic materials, such as magnetite. Inorganic magnetic materials also come into consideration, examples being metal oxides, metals such as cobalt for example, or mixtures and alloys of different metals, such as iron-platinum or iron-gold for example. The surfaces of these magnetizable particles can then be modified with the linker structures according to the invention.

The magnetic carrier materials can be present in a spherical or irregular form. Preferably, the diameter of the particles is in the range from few hundred nanometers to a few hundred micrometers. In addition to such microparticles, however, nanoparticles having a particle diameter of a few nanometers also come into consideration. The particles can have, for example, ferromagnetic, ferrimagnetic, paramagnetic, and also superparamagnetic properties.

Furthermore, nonwoven fabrics, for example, can also be employed as carrier materials. Functionalized nonwoven fabrics according to the invention can be, for example, intalled in spin columns. Preferred embodiments are described hereinafter.

Various methods are known in the prior art for functionalizing the surface of a carrier with, for example, SAMs, Langmuir-Blodgett films, or silane groups. Appropriate methods are described, for example, in Nixon et al. (Palladium Porphyrin Containing Zirconium Phosphonate Langmuir-Blodgett Films Chem. Mater 1999, 11, 965-976). It is advantageous to initially bind the linker structure having the phosphate or phosphonate group to the carrier before the immobilization of zirconium ions is effected. A multistage deposition method is preferred, which can also be applied analogously to the deposition of SAM structures and silanizations. Hereinafter, the deposition process is described on the basis of phosphonate alternatives.

Initially, a phosphonate layer is deposited on the carrier. Phosphonates have the structure RPO₃H₂. Group R corresponds to the flexible linker structure according to the invention, having at least 5, preferably more than 8, especially preferably more than 10 carbon atoms. Depending on the linker structure (Langmuir-Blodgett films, silanizations, or SAM structures), either covalent or noncovalent links to the surface of the carrier are formed.

After the phosphonate linker structure has been applied to the carrier, the coated carrier can be submerged in a zirconium solution (for example, ZrOCl₂) in order to immobilize zirconium ions on the phosphate group and, accordingly, to form the phosphopeptide-binding functional group. Alternatively, the zirconium solution can be applied to the sample field in order to achieve loading. The thus prepared carrier is then ready for usage in enriching phosphopeptides. A corresponding method applies when employing a phosphate linker structure.

For silanization, aminoalkylalkoxysilanes can, for example, be employed. In order to make a sufficiently flexible linker structure available, the aminoalkyl-alkoxysilane group preferably has one alkyl chain having at least 5, preferably more than 8, and especially preferably ≧10 carbon atoms. They subsequently form the actual linker structure. Carrier materials which have been functionalized with appropriate aminoalkylalkoxysilanes can, by treatment with POCl₃ for example, be subsequently provided with phosphonate groups and converted into phosphorus-containing groups (—N—P bonding). As the last step, zirconium ions are added in order to generate a PO₃Zr⁴⁺ group which binds phosphopeptides highly specifically.

Self-Assembled Monolayers (SAMs) are well-ordered, monomolecular films which offer great flexibility in that they also allow huge scope for variation. Appropriate films which can be employed according to the invention as linker structures can be formed, for example, from thiol compounds, for example omega-substituted alkanethiols and disulfides. Alkylthiols have the structure R—(CH₂)ₙ—SH, where SH represents the thiol head group. n can represent any number, depending on the desired length of the linker structure. Typically, n is between 5 and 21. R here corresponds to the terminal functional group, presently the —PO₄H₂ or —PO₃H₂ group. As explained, the phosphate or phosphonate group is functionalized with zirconium ions. As elucidated above, a polymer group, for example polyethylene glycol or another group, can also be incorporated in the alkyl chain or attached to the alkyl chain. A correspondingly functionalized linker structure enables enormous flexibility, which presently leads surprisingly to an improved enrichment of phosphopeptides. The fact that this can be achieved via the length and the structure of the linker structure is surprising. Not only thiol compounds but also, for example, dialkyl sulfides can be employed.

Not only alkanethiols but also, for example, dialkyl disulfides, dialkyl sulfides, and ethylene glycol alkanethiol derivatives can be employed as linker structures.

To functionalize the surface of the carrier, alkanethiols according to the formula (I), for example, can be used:

HS(CH₂)ₙX (I)

where
X=phosphate or phosphonate group
n=5 to 28, preferably 5 to 18, especially preferably 9 to 18.

In a further embodiment, dialkyl disulfides according to the formula (II) can also be employed:

X(CH₂)ₘS—S(CH₂)ₙX (II)

where
X=phosphate or phosphonate group
m=1 to 28, preferably 5 to 18, especially preferably 9 to 18;
n=1 to 28, preferably 5 to 18, especially preferably 9 to 18;
and n+m≧5.

In a further embodiment, dialkyl sulfides according to the formula (III) can be used:

X(CH₂)ₘS(CH₂)ₙX (III)

where
X=phosphate or phosphonate group
m=1 to 28, preferably 5 to 18, especially preferably 9 to 18;
n=1 to 28, preferably 5 to 18, especially preferably 9 to 18;
and n+m≧5.

In a further embodiment, ethylene glycol alkanethiol derivatives according to at least any of the formulae (IV)-(VI) can be employed:

HS(CH₂)ₘ(EO)ₙX (IV)

X(EO)ₙ(CH₂)ₘS—S(CH₂)ₘ(EO)ₙX (V)

X(EO)ₙ(CH₂)ₘS(CH₂)ₘ(EO)ₙX (VI)

where
X=phosphate or phosphonate group
m=1 to 28, preferably 5 to 18, especially preferably 9 to 18;
n=1 to 12, preferably 3 to 6;
and the groups defined by n+m have together at least 5 carbon atoms.

In a further embodiment, each of the compounds corresponding to the formulae (I) to (VI) can be used as a mixture of phosphate and phosphonate. Furthermore, compounds of different substance classes according to the formulae (I) to (VI) can also be used as a mixture of two or more compounds. Compounds of the formulae (I) to (VI) are preferably deposited in the form of SAMs. Metals can be used as suitable carriers.

SAMs are obtained, for example, by submerging the substrate, preferably a MALDI carrier, in a dilute solution of a thiolate-forming compound, for example in alkylthiols (see above). Hereinafter, a possible coating method is elucidated by means of an example (alkylthiol). However, any other compound which can form a thiolate layer can also be employed for the purposes of the present invention as a linker group.

The alkylthiol compounds (or other suitable linker structures, see above), owing to the thiol head group (SH), adsorb strongly to the substrate surface and form close-packed monolayers having extended hydrocarbon chains [—(CH₂)ₙ] or derivatives thereof. It is believed that the thiol head group, after chemisorption on the substrate, loses hydrogen to form a thiolate. Since the alkylthiolate compounds on the substrate surface are anchored via the sulfur head, the outwardly exposed surface of the SAM coating displays the terminal phosphate or phosphonate group which can be functionalized with zirconium ions. As a result, a surface is made available which is optimally configured for enriching phosphopeptides. This more particularly since the ordered structures enable optimal connections to varyingly sized and varyingly shaped peptides.

The present invention further makes available a kit for enriching phosphopeptides, characterized in that it includes a carrier according to the invention. Optionally, the kit may also include further components, for example binding and/or wash buffers. Details about the carriers and suitable linker structures have already been described in detail; we refer to the explanations above.

The invention and the advantages achieved with it will now be more particularly described with reference to examples. These are not restricting, but represent preferred embodiments of the present invention.

### EXAMPLE 1

The performance of the zirconium phosphonate chip according to the invention was compared with conventional IMAC chips (loaded with Fe(III) or Zr(IV)). The specificity and the phosphopeptide-binding preferences were tested with regard to a peptide mixture (Invitrogen) which has four phosphorylated and three unphosphorylated peptides and also an extra synthetic threonine-phosphorylated peptide (phosphorylated peptides: pS, pY, pT, pTpY). This mixture (100 fmol), together with 100 fmol of a standard BSA digest (Waters) which does not comprise any phosphopeptides, was applied to one spot on each chip. The IMAC ships were processed according to the instructions of the manufacturer and focused with DHB (1 mg per ml) as matrix. The chip according to the invention was treated as follows:
Washing the chip with 70% ACN (once)
Washing again with 50% ACN/300 mM acetic acid (once)
Equilibrating with 300 mM acetic acid (two times 2 minutes)
Loading the chip with 100 mM zirconium chloride (ZrCl₄, incubating for 20 minutes; alternatively, ZrOCl works as well)
Washing three times with 300 mM acetic acid
Adding the sample, diluted in 300 mM acetic acid for 20 minutes
Washing three times with 30% ACN/300 mM acetic acid
Eluting with DHB (1 mg/ml) in 1% H₃PO₄ and drying (alternatively, 0.5% H₃PO₄ can be used, accelerates drying)

All wash and loading solutions had a volume of 10 microliters; the elution solution had a volume of 2 microliters.

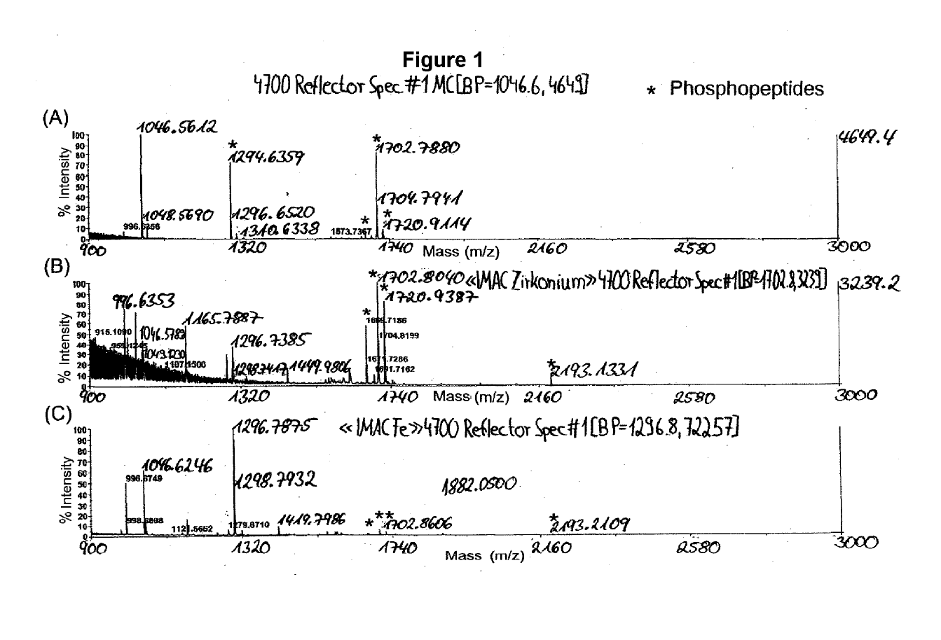

FIG. 1 shows, accordingly, the results achieved with different MALDI chips in enriching phosphopeptides by means of various: 100 fmol of a mix of five different phosphopeptides and numerous unphosphorylated peptides were studied on the following MALDI chips:

(a) a Zr-phosphonate chip according to the invention;

### (b) a Zr-loaded Mass Spec Focus Chip (QIAGEN) and

### (c) an Fe-loaded Mass Spec Focus Chip (QIAGEN)

The samples were applied and processed as described. Each of the enriched-phosphopeptide peaks is marked with an asterisk. It is clear that the Zr-phosphonate chip has a high specificity, since 4 out of 5 phosphopeptides were detected, but only one false positive unphosphorylated peptide was detectable. After phosphopeptide purification on each of the Mass Spec Focus Chips, 4 out of 5 phosphopeptides were likewise detected. Surprisingly, the selectivity here, however, is different, since these chip types had evidently not bound a peptide phosphorylated at threonine (1294 m/z), but a peptide phosphorylated at serine (2193 m/z). This in turn could not be detected with the help of the Zr-phosphate chip. At the same time, both Mass Spec Focus Chips, more particularly the Fe-loaded chip type, show, however, a lower specificity compared with the Zr-phosphonate chip, since a larger number of contaminating, unphosphorylated peptides had been coenriched here.

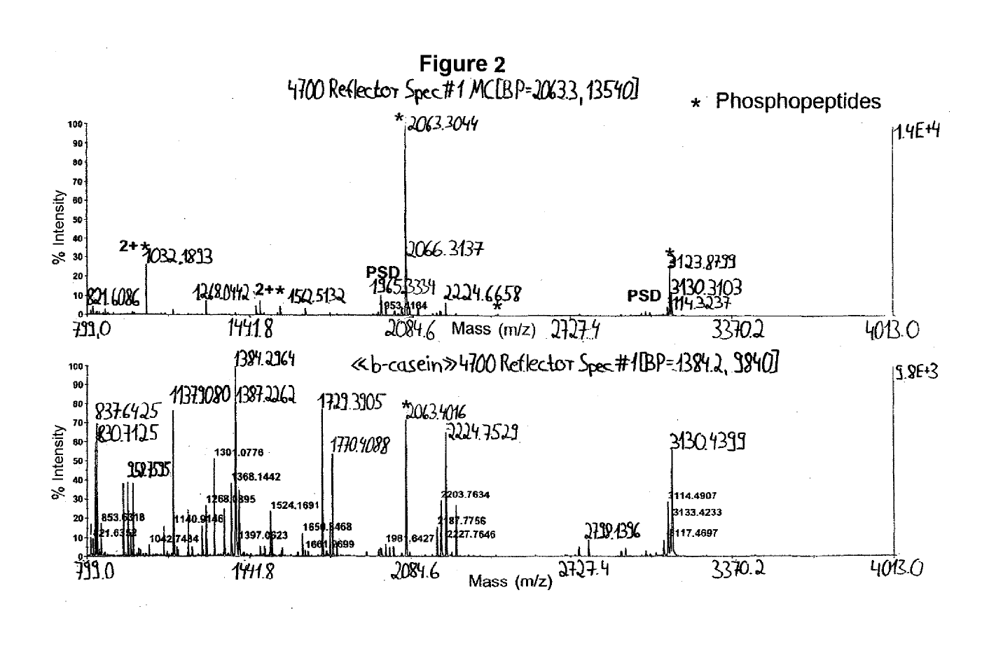

FIG. 2 illustrates the specificity of the Zr-phosphonate chip. The upper spectrum shows the results of the following experiment: beta-casein digest (2 pmol) was applied on the chip and processed as described above. The lower spectrum shows the results of the following experiment: beta-casein digest (2 pmol) was applied on a neighboring spot on the same chip, but none of the subsequent wash steps were carried out. Each of the phosphopeptide peaks is marked with an asterisk. “2+” indicates doubly charged ions in the spectrum, while “PSD” is used to mark post source decay fragments, which arise in the measurement during the ionization process by loss of phosphoric acid. From the comparison of the two spectra, the high purification efficiency and hence associated specificity of the Zr-phosphonate chip is apparent. The beta-casein digest employed has a high number of peptide peaks in the spectrum (lower spectrum) which, apart from the binding phosphopeptides, could be almost completely washed off (upper spectrum).

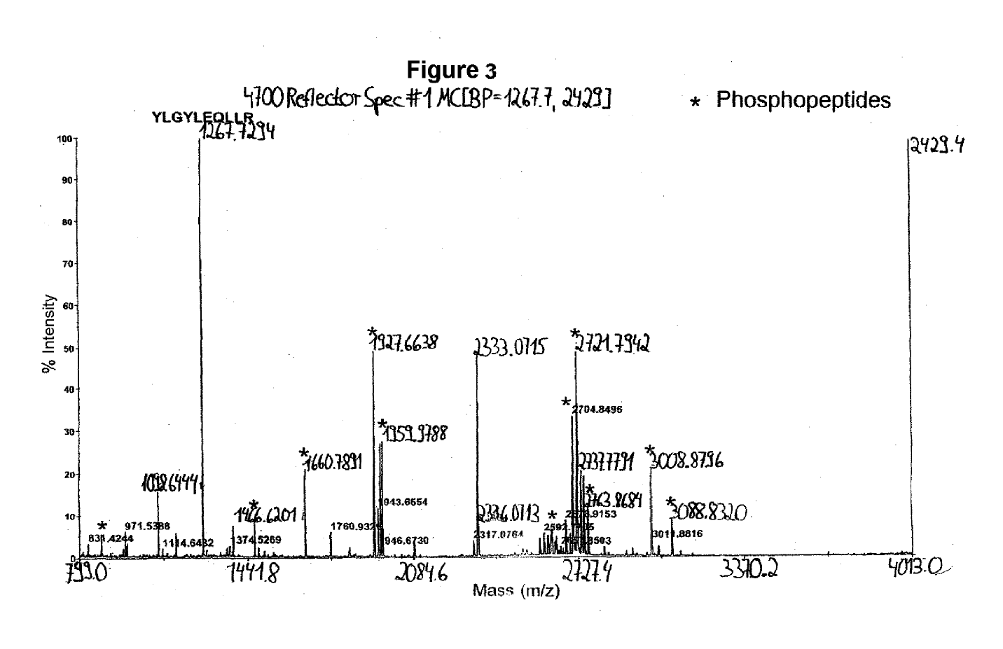

FIG. 3 shows the spectrum of an alpha-casein digest (2 pmol) after processing on the Zr-phosphonate chip. The commercially available alpha-casein has a multiplicity of phosphorylation sites which, after digestion and processing, were detected on the chip. At the same time, the high specificity of the chip is also apparent here, since, in addition to the high number of phosphopeptides, only three unphosphorylated peptides were detectable. This spectrum was likewise used for the comparison between the phosphopeptides purified in Zhou et al. (Zirconium phosphonate-modified porous silicon for highly specific capture of phosphopeptides and MALDI-TOF MS analysis, J. Prot. Res., 2006, 5, 2431-2437) and the phosphopeptides found here (see table 1).

Table 1 shows a comparison of the detected phosphopeptide peaks from an alpha-casein digest after processing on a Zr-phosphonate chip according to the invention with the phosphopeptides found by Zhou et al. Zhou et al. likewise carried out the purification of phosphopeptides on a Zr-phosphonate-functionalized carrier for MALDI-TOF analyses, which differs, however, in the type of linker structure used. The comparison reveals that, compared with the methodology described in Zhou et al., the technology according to the invention enabled enrichment and detection of a higher number of phosphopeptides in the same application (alpha-casein digest).

| Exp. mass | Described in |
|---|---|
| [M + H] + Da | Zhou et al. |
| 880.72 (loaded 2 times) | — |
| 1466.97 | X |
| 1539.96 | X |
| 1661.19 | X |
| 1833.28 | X |
| 1848.16 | X |
| 1928.14 | X |
| 1944.13 | — |
| 2592.77 | — |
| 2619.45 | X |
| 2678.57 | — |
| 2703.86 | — |
| 2720.53 | X |
| 2746.87 | — |
| 2762.85 | — |
| 2935.71 | X |
| 3008.61 | X |
| 3088.57 | X |

(“X” = detected/“—” = not detected)

### EXAMPLES 2 and 3

A phosphorylated alkylthiol in the form of a monolayer was applied on a glass fiber nonwoven fabric sputtered with gold. This nonwoven fabric can be installed in spin columns and serve to enrich phosphopeptides.

Furthermore, gold particles were cleaned in a low-pressure plasma and modified by application of the same functionalized linker structures. These particles were deposited in a spin column on an inert membrane material and likewise serve to enrich phosphopeptides.

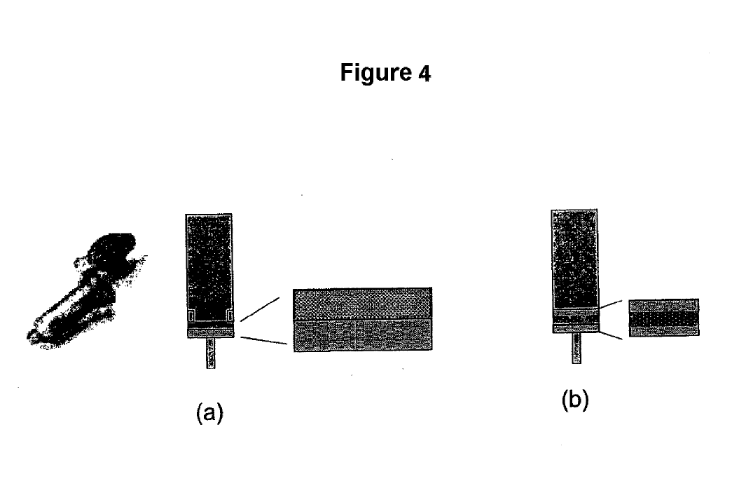

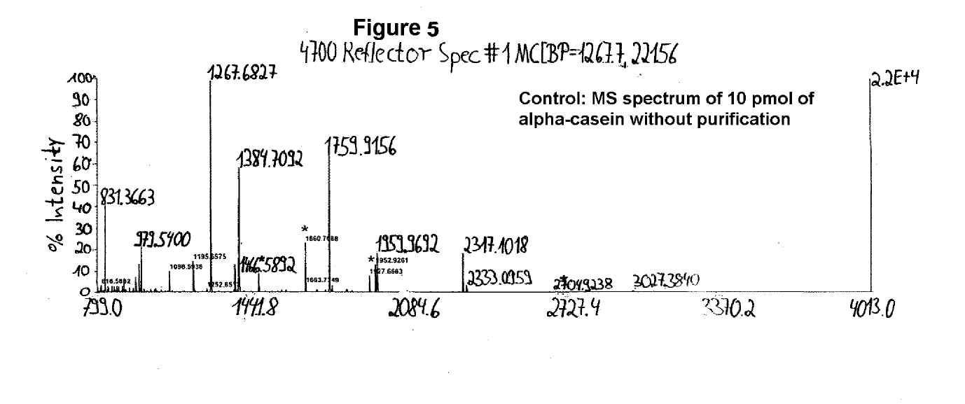

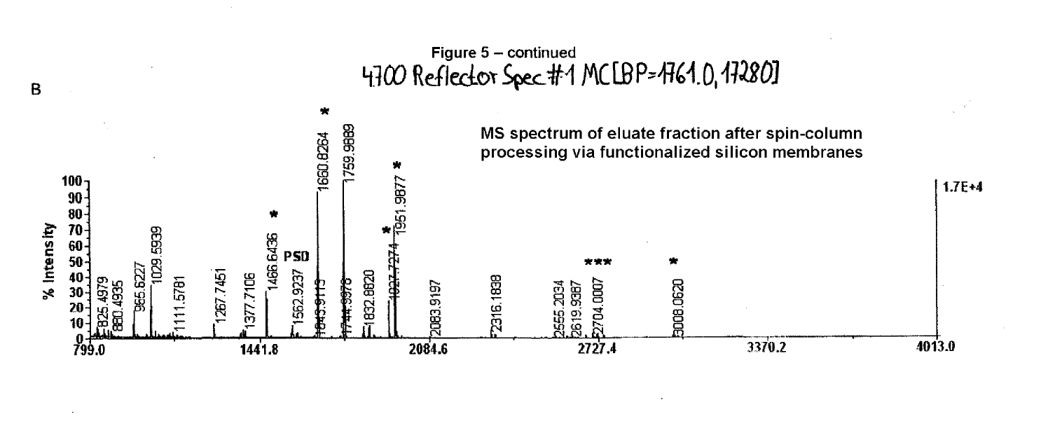

FIG. 4 shows schematically the structure of the spin column body. In the first variant (a), a functionalized nonwoven fabric was employed. For this purpose, a silica membrane was sputtered with gold and functionalized with the linker structures according to the invention. A Vyon frit was also employed. In variant (b), functionalized gold particles were employed. The particles are preferably smaller than 45 μm. Preferably, the gold particles are held in a sandwich, for example, with the following construction: frit (for example, Vyon frit), functionalized gold particles, filter membrane, and frit (for example, Vyon frit). These spin columns can be employed for purifying phosphopeptides.

These spin columns were processed according to the following protocol:
Activating the chromatographic material by applying and subsequently centrifuging 250 μl of acetic acid (300 mM)
Loading the column with 100 mM zirconium oxychloride (ZrOCl₂), incubating for 10 minutes, centrifuging (spin method)
Washing three times with 250 μl of acetic acid (300 mM) in the spin method
Adding the sample (50 μl), diluted in 30% ACN/300 mM acetic acid for 20 minutes, centrifuging
Washing three times (250 μl) with 30% ACN/300 mM acetic acid
Eluting by adding 50 μl of ammonia solution (25%), incubating for 5 minutes, and subsequently incubating
Acidifying the sample by adding finally 1% formic acid
Applying the sample on a MALDI target or, optionally, prior concentrating of the sample by means of reversed-phase resin (ZipTip)

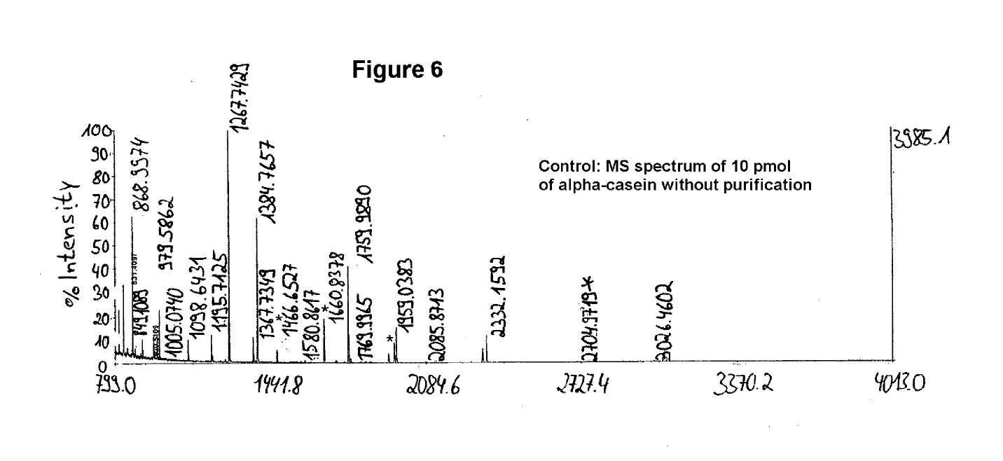

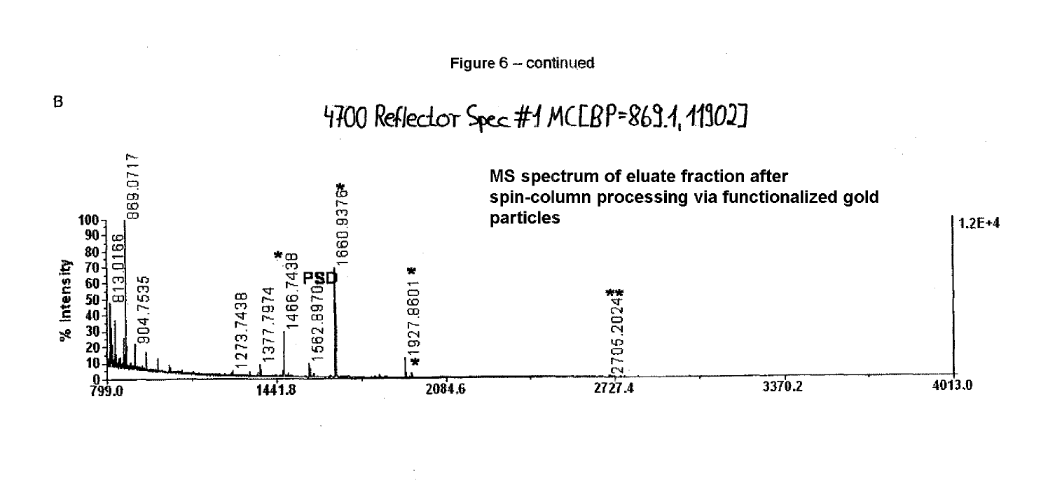

FIGS. 5 and 6 show spectrums of 10 pmol of alpha-casein before and after processing in a spin column in which either a functionalized nonwoven fabric was installed (see FIG. 5) or functionalized gold particles were deposited (see FIG. 6).

1. A method for enriching phosphopeptides, the method comprising:
providing a carrier which carries on its surface phosphate and/or phosphonate groups which are functionalized with zirconium ions, wherein the phosphate and/or phosphonate groups functionalized with zirconium ions are bound to the carrier via linker structures and the linker structures have at least one alkyl chain which has at least 5 carbon atoms; and
using the carrier to enrich phosphopeptides.

2. The method as claimed in claim 1, wherein the linker structures have an alkyl chain which has ≧10 carbon atoms.

3. The method as claimed in claim 1, wherein the linker structures have at least one or more of the following features:
a) at least some of the linker structures have at least one inert polymer group, which is integrated in the alkyl chain or attached to the alkyl chain; and/or
b) at least some of the linker structures are selected from the group consisting of alkanethiols, dialkyl sulfides, and ethylene glycol alkanethiol derivatives; and/or
c) at least some of the linker structures are formed from alkanethiols according to the formula (I):
HS(CH₂)ₙX (I)
where
X=phosphate or phosphonate group
n=5 to 28, preferably 9 to 18; and/or
d) at least some of the linker structures are formed from dialkyl disulfides according to the formula (II):
X(CH₂)ₘS—S(CH₂)ₙX (II)
where
X=phosphate or phosphonate group
m=1 to 28, preferably 9 to 18;
n=1 to 28, preferably 9 to 18;
and n+m≧5; and/or
e) at least some of the linker structures are formed from dialkyl sulfides according to the formula (III):
X(CH₂)ₘS—S(CH₂)ₙX (III)
where
X=phosphate or phosphonate group
m=1 to 28, preferably 9 to 18;
n=1 to 28, preferably 9 to 18;
and n+m≧5; and/or
f) at least some of the linker structures are formed from ethylene glycol alkanethiol derivatives according to at least one of the formulae (IV)-(VI):
HS(CH₂)ₘ(EO)ₙX (IV)
X(EO)ₙ(CH₂)ₘS—S(CH₂)ₘ(EO)ₙX (V)
X(EO)ₙ(CH₂)ₘS(CH₂)ₘ(EO)ₙX (VI)
where
X=phosphate or phosphonate group
m=1 to 28, preferably 9 to 18;
n=1 to 12, preferably 3 to 6;
and the groups defined by n+m have together at least 5 carbon atoms.

4. The method as claimed in claim 1, wherein the carrier is a plate, a filter, a small column, a nonwoven fabric, a particle, a magnetic particle, a polymer particle, a metal particle, a MALDI carrier, and/or a silica carrier.

5. The method as claimed in claim 1, wherein the linker structures are bound to the carrier by covalent or noncovalent bonding.

6. The method as claimed in claim 1, wherein the linker structures are formed by silanization, self-assembled monolayer films or Langmuir-Blodgett films.

7. The method as claimed in claim 6, wherein linker structures formed by Langmuir-Blodgett films are bound to the carrier via ionic or electrostatic bonds, linker structures formed by self-assembled monolayer films are bound to the carrier via SH groups or disulfide groups, and linker structures formed by silzanization are bound to the carrier by covalent bonding.

8. A carrier for enriching phosphopeptides that carries on its surface phosphate and/or phosphonate groups which are functionalizable with zirconium ions, wherein the phosphate and/or phosphonate groups are bound to the carrier via linker structures and the linker structures have at least one alkyl chain which has at least 5 carbon atoms.

9. The carrier as claimed in claim 8, wherein the linker structures have at least one or more of the following features:
a) at least some of the linker structures have at least one inert polymer group; and/or
b) at least some of the linker structures have at least one inert polymer group, which is integrated in the alkyl chain and/or attached to the alkyl chain; and/or
c) at least some of the linker structures are selected from the group consisting of alkanethiols, dialkyl sulfides, and ethylene glycol alkanethiol derivatives; and/or
d) at least some of the linker structures are formed from alkanethiols according to the formula (I):
HS(CH₂)ₙX (I)
where
X=phosphate or phosphonate group
n=5 to 28, preferably 9 to 18; and/or
e) at least some of the linker structures are formed from dialkyl disulfides according to the formula (II):
X(CH₂)ₘS—S(CH₂)ₙX (II)
where
X=phosphate or phosphonate group
m=1 to 28, preferably 9 to 18;
n=1 to 28, preferably 9 to 18;
and n+m≧5; and/or
f) at least some of the linker structures are formed from dialkyl sulfides according to the formula (III):
X(CH₂)ₘS(CH₂)ₙX (III)
where
X=phosphate or phosphonate group
m=1 to 28, preferably 9 to 18;
n=1 to 28, preferably 9 to 18;
and n+m≧5; and/or
g) at least some of the linker structures are formed from ethylene glycol alkanethiol derivatives according to at least one of the formulae (IV)-(VI):
HS(CH₂)ₘ(EO)ₙX (IV)
X(EO)ₙ(CH₂)ₘS—S(CH₂)ₘ(EO)ₙX (V)
X(EO)ₙ(CH₂)ₘS(CH₂)ₘ(EO)ₙX (VI)
where
X=phosphate or phosphonate group
m=1 to 28, preferably 9 to 18;
n=1 to 12, preferably 3 to 6;
and the groups defined by n+m have together at least 5 carbon atoms.

10. The carrier as claimed in claim 8, wherein the linker structures are bound to the carrier by covalent or noncovalent bonding.

11. The carrier as claimed in claim 8, wherein it is functionalized with zirconium ions and, optionally, comprises bound phosphopeptides.

12. A method for enriching phosphopeptides comprising:
providing a carrier as claimed in claim 8; and
using the carrier to enrich phosphopeptides.

13. A method for producing a carrier as claimed in claim 8, comprising:
binding the surface of the carrier with phosphate and/or phosphonate groups via linker structures which have at least one alkyl chain having at least 5 carbon atoms.

14. The method as claimed in claim 13, wherein the carrier having phosphate and/or phosphonate groups is brought into contact with zirconium ions to generate on the carrier a phosphopeptide-binding, functional surface.

15. A kit for enriching phosphopeptides that includes a carrier as claimed in claim 8.

16. The method as claimed in claim 3 wherein the at least one inert polymer group is polyethylene glycol.

17. The method as claimed in claim 9 wherein the at least one inert polymer group is polyethylene glycol.
